(19) United States
(12) Patent Application Publication
(10) Pub. No.: US 2016/0361154 A1
(43) Pub. Date: Dec. 15, 2016

(54) CLOSURE DEVICE

(71) Applicants: Gregory C. SAMPOGNARO, Monroe, LA (US); Stephen RAMEE, New Orleans, LA (US); Gwendolyn A. WATANABE, Los Altos Hills, CA (US); Jeffery A. KROLIK, Campbell, CA (US)

(72) Inventors: Gregory C. SAMPOGNARO, Monroe, LA (US); Stephen RAMEE, New Orleans, LA (US); Gwendolyn A. WATANABE, Los Altos Hills, CA (US); Jeffery A. KROLIK, Campbell, CA (US)

(21) Appl. No.: 15/249,071

(22) Filed: Aug. 26, 2016

Related U.S. Application Data
(63) Continuation of application No. 12/938,218, filed on Nov. 2, 2010, now abandoned.
(60) Provisional application No. 61/280,389, filed on Nov. 3, 2009.

(51) Int. Cl. A61F 2/07; A61F 2/844; A61F 2/89; A61B 17/00

(52) U.S. Cl. CPC A61F 2/07; A61B 17/0057; A61F 2/844; A61F 2/89; A61F 2002/061

(57) ABSTRACT

Described here are devices and methods for closing one or more vascular openings. The devices may include a stent graft comprising a stent framework and a graft material at least partially covering the stent framework. The stent framework may comprise one or more axial segments, and at least one of the axial segments may comprise an access port through which a catheter or treatment device may enter the stent graft. The methods may comprise occluding blood flow upstream of a vascular opening, and delivering a closure device to block, cover, or seal the vascular opening.

### CROSS REFERENCE TO RELATED APPLICATIONS

This application is a continuation of U.S. application Ser. No. 12/938,218, filed Nov. 2, 2010, which claims priority to U.S. Provisional Application No. 61/280,389, filed Nov. 3, 2009, both of which are hereby incorporated by reference in their entirety.

### FIELD

The devices and methods described herein are in the field of vascular closure.

### BACKGROUND OF THE INVENTION

Endovascular procedures are an increasingly common alternative to open surgical procedures. Conducted from the interior of a blood vessel, endovascular procedures can be performed under local anesthesia with no (or partial) cardiac bypass, and require a shorter hospitalization than open surgical procedures. Prior to or during an endovascular procedure, access to the vasculature is obtained via one or more arteriotomies or other openings formed in the wall of a blood vessel, and one or more catheters or other treatment devices may be advanced therethrough into the vasculature.

Some endovascular procedures, especially those designed to treat the heart or large blood vessels such as the aorta, may require large-French vascular access. For example, treatment devices used in endovascular aneurysm repair procedures (treating abdominal or thoracic aortic aneurysms by delivery of a stent graft or other graft thereto) and endovascular aortic valve replacement generally range in size from about 12 Fr (about 4 mm) to about 30 Fr (about 10 mm). Accordingly, any vascular access point (e.g., the arteriotomy or other vessel opening) must be large enough to accommodate these large-French treatment devices, and thus vascular access is usually obtained through the common femoral artery or one of the iliac arteries (e.g., the common iliac artery, the external iliac artery, or the internal iliac artery). Manual pressure is usually insufficient to close such large-French openings, and instead these openings are typically closed using one or more sutures or suture-based devices. This generally requires the presence of a surgeon in an operating room, and often requires placing the patient under general anesthesia. Accordingly, it may be desirable to provide improved methods of closing large-French vessel openings in a manner that does not require the presence of a surgeon.

### BRIEF SUMMARY OF THE INVENTION

Described here are devices and methods for closing one or more openings in a vessel wall. In some variations of the devices described here, the device comprises a stent graft, wherein the stent graft comprises a stent framework comprising a first axial segment and a graft material at least partially covering the stent framework, and wherein the first axial segment comprises a first access port in a side of the stent framework, and wherein the first access port is sized and configured to receive a treatment device therethrough. In some of these variations the first axial segment may comprise a first saddle-shaped ring, and wherein the first saddle-shaped ring defines the first access port. In some of these variations, the first axial segment comprises a second saddle-shaped ring, and wherein the second saddle-shaped ring defines a second access port. In other variations, the stent framework may comprise a second axial segment, wherein the second axial segment comprises at least one access port. In some of these variations, the second axial segment may comprise at least one saddle-shaped rings. The access ports may be sized and shaped to receive any suitable treatment devices or catheters. In some variations, the first access port may be sized and shaped to receive a treatment device having a diameter of at least about 5 French therethrough. In other variations, the first access port may be sized and shaped to receive a treatment device having a diameter of at least about 7 French therethrough. In other variations, the first access port may be sized and shaped to receive a treatment device having a diameter of at least about 8 French therethrough. In still other variations, the first access port may be sized and shaped to receive a treatment device having a diameter of at least about 15 French therethrough. In yet other variations, the first access port may be sized and shaped to receive a treatment device having a diameter of at least about 20 French therethrough. In some of these variations, the first access port may be sized and shaped to receive a treatment device having a diameter of about 22 French therethrough.

The stent grafts may comprise any suitable material or materials. In some variations, the stent framework may comprise a nickel-titanium alloy or other shape memory alloy. In other variations, the stent framework may comprise one or more biodegradable polymers. In some variations, the graft material may comprise polytetrafluoroethylene or expanded polytetrafluoroethylene. In some of these variations, the stent framework may be cut from a tubular piece of material. In some of these variations, the stent framework may be laser cut from the tubular piece of material. The stent graft may have any suitable dimensions. In some variations, the stent graft may have a diameter at least about 6 mm. In other variations, the stent graft may have a diameter greater at least about 7 mm.

In some variations, the stent framework may comprise a second axial segment at a first end of the stent framework, and a third axial segment at a second end of the stent frame. In some of these variations, the stent framework may be sized and shaped such that the second axial segment is configured to engage the blood vessel upstream of the opening and such that the third axial segment is configured to engage the blood vessel downstream of the opening. In some of these variations the stent framework may comprise a fourth axial segment positioned between the second and third axial segments. In some of these variations, the fourth axial segment may comprise one or more access ports.

The graft material may cover any suitable portion of the stent framework. In some variations, the graft material may entirely cover an outer surface of the stent framework. In some variations where the stent framework comprises a first axial segment, a second axial segment at a first end of the stent framework and a third axial segment at a second end of the stent framework, the graft material may cover an outer surface of the first axial segment, partially cover an outer surface of the second axial segment, and partially cover an outer surface of the third axial segment. In variations where one or more axial segments comprises an access port, the graft material may cover all or some of the access port. In variations where the graft material covers an access port, entry into the stent graft through access port may comprise puncturing, piercing, or otherwise penetrating the graft material.

Also described here are methods of closing one or more blood vessels. In some variations, a method of closing an opening in the common femoral artery, external iliac artery, internal iliac artery, or common iliac artery, may comprise advancing a introducer sheath to a position upstream of the opening, wherein the introducer sheath comprises an expandable member, expanding the expandable member to occlude blood flow past the expandable member, and advancing a delivery catheter through the introducer sheath to a position near the opening; and delivering a closure device to close the opening. In some variations, the method may further comprise introducing a dilator into a contralateral femoral artery and advancing the dilator into the common iliac artery, and wherein advancing the introducer sheath comprises advancing the introducer sheath over the dilator. In some variations, the closure device may comprise a stent graft, wherein the stent graft may comprise a stent framework having a first axial segment and a graft material at least partially covering the stent framework, and wherein the first axial segment may comprise an access port in a side of the stent framework, the first access port is sized and configured to receive a treatment device therethrough.

The expandable member may be expanded in any suitable blood vessel. In some variations, expanding the expandable member may comprise expanding the expandable member in the common iliac artery. In some variations, expanding the expandable member may comprise expanding the expandable member in the external iliac artery. In some variations, expanding the expandable member may comprise expanding the expandable member in the common femoral artery. Additionally, in some variations, the method may comprise introducing the introducer sheath into a brachial artery.

In other variations, methods for closing an opening in a blood vessel, the opening having a treatment device placed therethrough, may comprise partially withdrawing the treatment device from the blood vessel, advancing an introducer sheath to a position upstream of the opening, the introducer sheath comprising an expandable member expanding the expandable member to occlude blood flow through the blood vessel, removing the treatment device from the blood vessel; and delivering a closure device to the blood vessel to close the opening. In some of these methods, the blood vessel is the common femoral artery.

In some of variations of these methods advancing the introducer sheath may comprise advancing the introducer sheath through a contralateral femoral artery. In other variations, delivery of the closure device may comprise advancing a delivery catheter through the introducer sheath, and delivering the closure device from the delivery sheath. In some of these variations, the closure device may comprise a stent graft, the stent graft comprising a stent framework having a first axial segment and a graft material at least partially covering the stent framework, and wherein the first axial segment comprises an access port in a side of the stent framework, the first access port is sized and configured to receive a treatment device therethrough. In some of these variations, the method further comprises aligning the stent graft such that the first access port is placed adjacent to the opening.

### BRIEF DESCRIPTION OF THE DRAWINGS

FIGS. 1A-1C depict a perspective view, a front view, and a side view, respectively, of one variation of the devices described here. FIG. 1D depicts the device of FIGS. 1A-1C placed in a blood vessel.

FIG. 2 depicts an illustrative variation of an axial portion of a stent framework suitable for use with the devices described here.

FIGS. 3A and 3B depict a side view and a front view, respectively, of an illustrative variation of an axial portion of a stent framework suitable for use with the devices described here.

FIG. 4 shows an illustrative depiction of some of the major arteries of the abdomen and legs.

FIGS. 5A, 5B, 5C, 5D and 5E depict an illustrative method of closing an opening in a blood vessel.

FIGS. 6A and 6B depict two front views of an illustrative variation of the devices described here.

FIG. 7 depicts a side view of an illustrative variation of a stent framework suitable for use with the devices described here.

FIGS. 8A, 8B, and 9 depict illustrative variations of axial portions of stent frameworks suitable for use with the devices described here.

### DETAILED DESCRIPTION OF THE INVENTION

Described here are devices and methods for closing one or more openings in a vessel wall. Specifically, the devices described here comprise one or more stent grafts. The stent graft may comprise a graft material and a generally-cylindrical stent framework having one or more axial segments. At least one of the axial segments may define an access port in a side of the stent framework, which may allow a catheter or treatment device to be inserted through a side of the stent graft.

Also described here are methods for closing one or more openings in a blood vessel. Generally these methods comprise placing an introducer sheath comprising a balloon or other expandable member upstream of the vascular opening, and temporarily occluding blood flow with the balloon. In variations where the vascular opening is in the common femoral artery, placing the introducer sheath may comprise inserting the introducer sheath into the contralateral common femoral artery, and advancing the introducer sheath up the contralateral iliac artery and into the ipsilateral iliac artery. A delivery catheter or other device may be advanced through the introducer sheath, and may be used to close the vascular opening. In some variations, this comprises delivering one or more stent grafts, such as the stent grafts described herein throughout, adjacent the opening to close or otherwise seal off the opening. In some instances, the methods described here may be utilized to close or seal one or more large-French openings (e.g., greater than about 10 Fr (about 3.3 mm)).

### Devices

The devices described here may comprise one or more stent grafts, which may comprise a stent framework and a graft material at least partially covering the stent framework. The stent grafts are generally cylindrical in shape, and may define one or more lumens along the longitudinal axis of the stent graft, such that blood may flow therethrough when the stent graft is placed in a blood vessel. The stent framework of the stent grafts described here generally comprise one or more axial segments, where at least one of the axial segments defines an access port, which may allow for re-entry into a vessel through a side of the stent graft, as will be described in more detail below. The stent grafts described here are generally expandable between a low-profile delivery configuration and an expanded deployed configuration for apposition against tissue. The stent grafts may be self-expandable, or may be expanded by a balloon or other expandable structure.

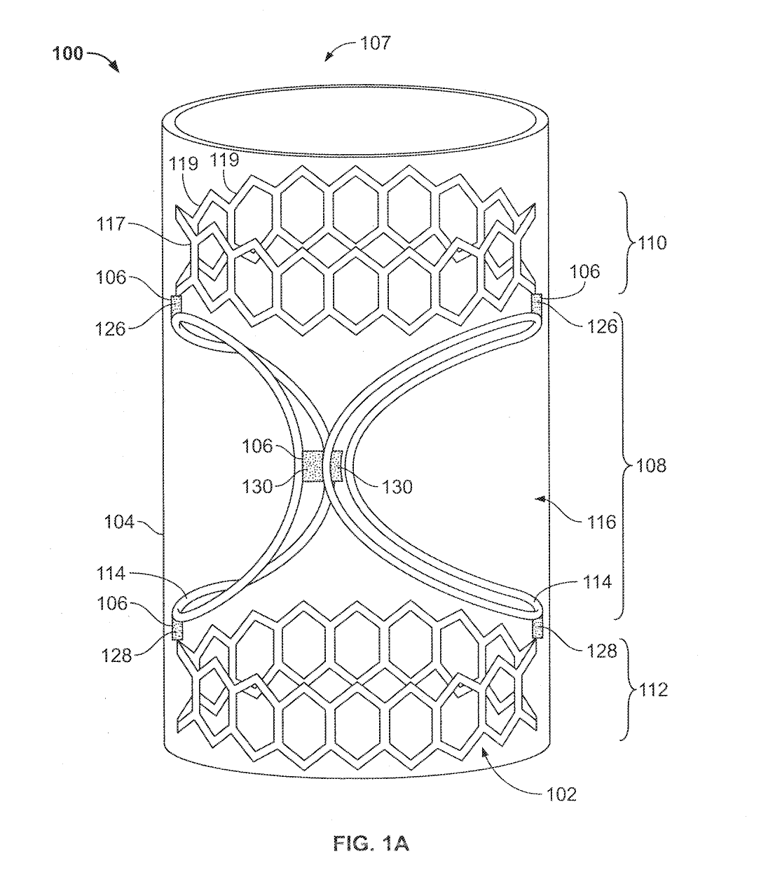

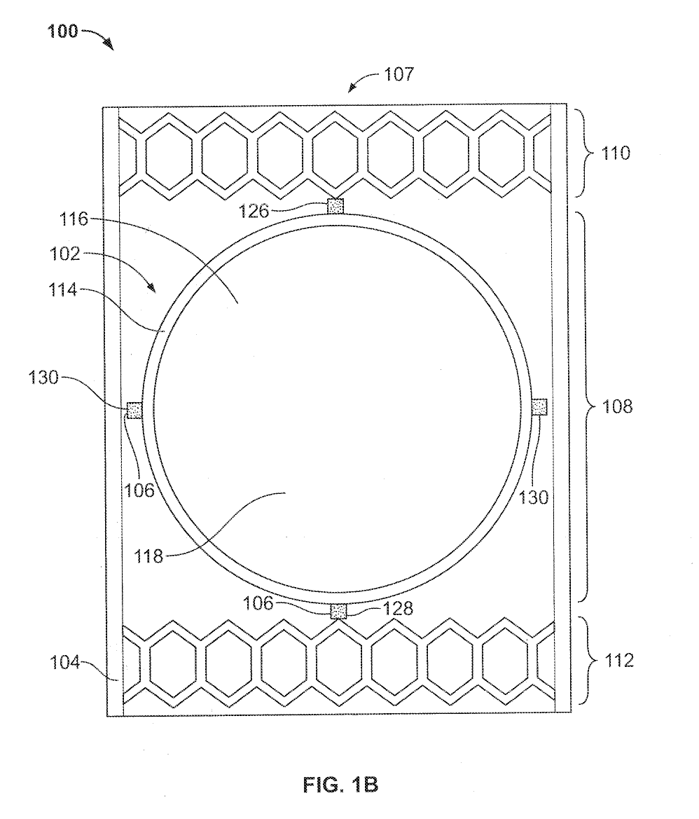

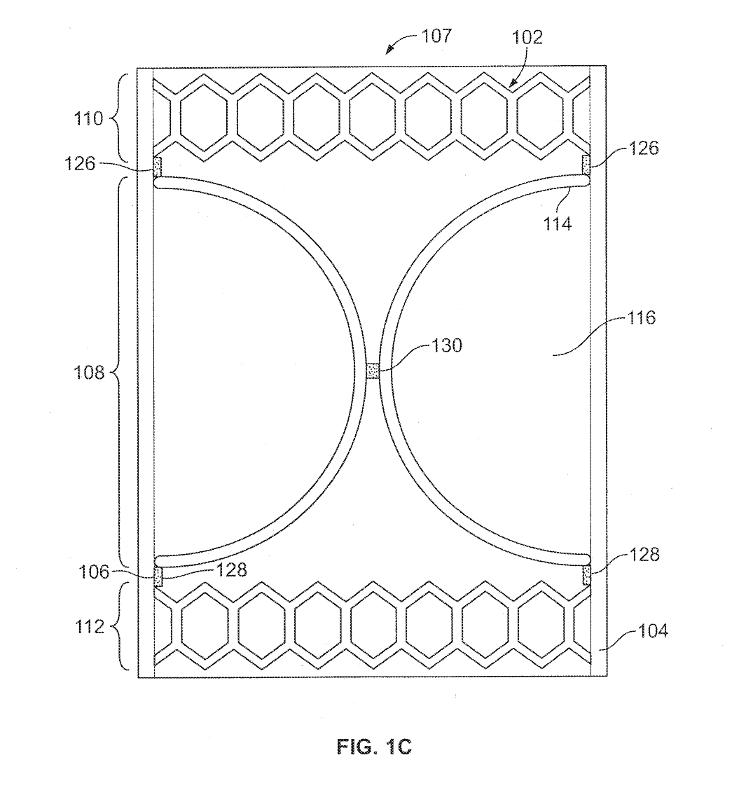

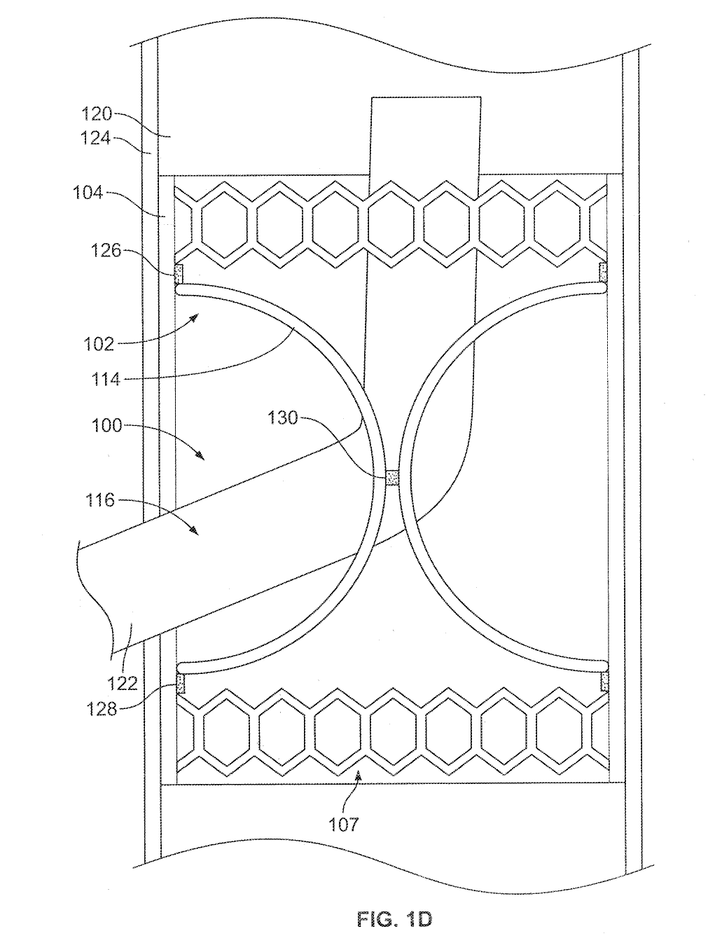

FIGS. 1A-1D illustrate one variation of stent graft (100). Specifically, FIGS. 1A-1C show a perspective view, front view, and side, respectively, of stent graft (100). As shown there, stent graft (100) may comprise a stent framework (102) and a graft material (104). Also shown there are a plurality of markers (106) overlaying (or otherwise attached to) different portions of the stent framework (102). Stent graft (100) may be generally cylindrical, defining a lumen (107) therethrough, such that blood may flow through lumen (107) when stent graft (100) is placed in a blood vessel. It should be appreciated that lumen (107) may be divided up into one or more sub lumens (not shown).

As shown in FIGS. 1A-1C, the cylindrical stent framework (102) may comprise a first axial segment (108) comprising two saddle-shaped rings (114), second axial segment (110) comprising a stent member (117) having a plurality of expandable cells (119), and third axial segment (112) a stent member (117) having a plurality of expandable cells (119). While shown in FIGS. 1A-1C as comprising three axial segments the stent frameworks of the stent grafts described here may comprise any suitable number of axial segments. For example, in some variations, the stent framework may comprise a single axial segment. In other variations, the stent framework may comprise two, three, four, five, or six or more axial segments. Each axial segment may serve one or more useful functions. In some instances, an axial segment may define one or more access ports, through which a needle, catheter, or other treatment device may be advanced to provide access to a blood vessel, as will be described in more detail below. Additionally or alternatively, an axial segment may act to support a portion of the graft material and/or a vessel wall. Additionally or alternatively, an axial segment may act to help anchor or otherwise hold the stent graft in place relative to a blood vessel. For example, when stent graft (100) described above in relation to FIGS. 1A-1C is used to close a vascular opening, second axial segment (110) may be configured to anchor the stent graft (100) upstream (or downstream) of the vascular opening (e.g., by expanding and engaging a portion of the blood vessel upstream of the opening). Similarly, the third axial segment (112) may be configured to anchor the stent graft (100) downstream (or upstream) of the vascular opening.

Each axial segment of the stent framework may have any suitable configuration. At least one of the axial segments may comprise one or more access ports. Generally, an access port provides an aperture or space in a side of the stent framework through which a needle, catheter, or other treatment device may be inserted without dislodging, damaging, or permanently deforming the stent frame. For example, in the variation of stent graft (100) described above in relation to FIGS. 1A-1C, each of the saddle-shaped rings (114) may comprise an access port (116). When viewed from the front, as shown in FIG. 1B, the saddle-shaped ring (114) may define an aperture (118) through a side of the stent framework (102). While shown in FIG. 1B as being generally circular, the aperture (118) may have any suitable shape (e.g., oval, square, rectangular, peanut-shaped, or the like).

The aperture (118) of the saddle-shaped ring (114) may act as an access port (116) in a side of stent graft (100) through which one or more needles, catheters, or treatment devices may be inserted. For example, in some instances stent graft (100) may be placed inside of a blood vessel (120) (e.g., the common femoral artery), as shown in FIG. 1D. It may then be desirable to gain subsequent access to a portion of blood vessel (120) occupied by stent graft (100) (e.g., to perform an additional endovascular procedure therethrough). Accordingly, a catheter (122) (or other treatment device) may be inserted into the blood vessel (120) through the vessel wall (124), access port (116) and into the lumen (107) of stent graft (100). In variations where graft material (104) overlays the access port (116) (and in variations where the graft material (104) is biodegradable, and has not yet biodegraded), the catheter (122) may pierce, puncture, or otherwise pass through the graft material (104). The catheter (122) may then be advanced out of the stent graft (100) through the blood vessel (120), and may be advanced to a target location in the vasculature.

Each access port of the stent grafts described here may be partially or fully covered by the graft material, as will be described in more detail below. For example, in the variation of stent graft (100) described above in relation to FIGS. 1A-1D, graft material (104) may fully cover the two access ports (116). Additionally, each access port may be configured to accept needles, catheters, or treatment devices of any suitable size and shape. In some variations an access port may be sized and shaped such that it may receive a treatment device or catheter of at least about 5 French (about 1.67 mm) therethrough. In other variations, the access port is sized and shaped to receive a treatment device of at least about 6 French (about 2 mm) therethrough. In still other variations, the access port is sized and shaped to receive treatment device of at least about 7 French (about 2.3 mm) therethrough. In yet other variations, the access port is sized and shaped to receive treatment device of at least about 8 French (about 2.7 mm) therethrough. In still other variations, the first access port may be sized and shaped to receive a treatment device having a diameter of at least about 15 French therethrough. In yet other variations, the first access port may be sized and shaped to receive a treatment device having a diameter of at least about 20 French therethrough. In some of these variations, the first access port may be sized and shaped to receive a treatment device having a diameter of about 22 French therethrough. In each of these variations, the access port may be configured to receive the treatment device without dislodging, moving, damaging or otherwise deforming the stent framework.

The stent framework may comprise any number of access ports (e.g., one, two, three, four, five, or six or more). In variations where the stent framework has a single access port, one axial segment may comprise a single access port. In variations where the stent framework comprises multiple access ports, a single axial segment may comprise all of the access ports, or multiple axial segments may comprise one or more access ports. For example, in the variation of stent graft (100) described above in FIGS. 1A-1D, first axial segment (108) of stent framework (102) comprises two access ports (116). Specifically, each of the saddle-shaped rings (114) of the first axial segment (108) defines an access port (116). In other variations, two axial segments of a stent framework each comprise a single access port. In still other variations, two axial segments each comprise two or more access ports. In yet other variations, three or more axial segments each comprise one or more access ports.

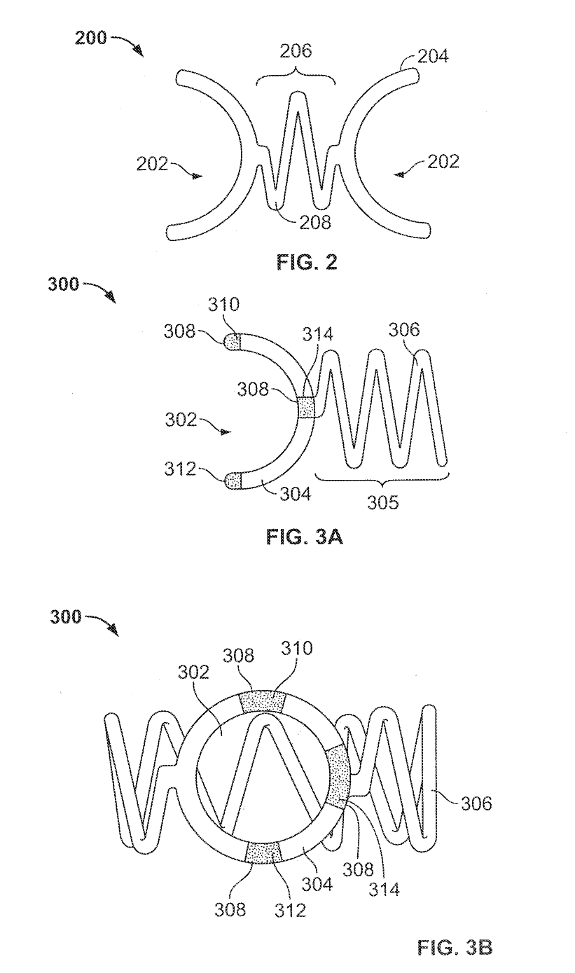

FIG. 2 shows a side view of a variation of axial segment (200) comprising two access ports (202). Specifically, axial segment (200) may comprise two saddle-shaped rings (202), which are connected via two expandable portions (206). While the expandable portions (206) shown in FIG. 2 as comprising a strut (208) with a zigzag pattern, it should be appreciated that any suitable expandable portion may connect the two saddle-shaped rings (202) (e.g., via strut comprising a meandering pattern, one or more expandable cells, or the like). While shown in FIG. 2 as comprising two saddle-shaped rings (202), it should be appreciated that an axial segment may comprise three or more saddle-shaped rings, each defining an access port. In these variations, the saddle-shaped rings may be connected in any suitable manner (e.g., directly connected, connected via one or more struts, one or more expandable portions, or the like).

FIGS. 3A and 3B show a side view and a front view, respectively, of a variation of an axial segment (300) comprising a single access port (302). As shown there, axial segment (300) may comprise a single saddle-shaped ring (304) defining the access port (302). Also shown there is a semi-cylindrical portion (305) connecting the sides of the saddle-shaped ring (304) and comprising a strut (306), and markers (308) overlaying portions of the saddle-shaped ring (304). Although shown in FIGS. 3A-3B as comprising a single strut (306), the semi-cylindrical portion (305) may comprise two or more struts, or a plurality of expandable cells. Additionally, while strut (306) is shown in FIGS. 3A-3B as having a zigzag pattern that may be capable of expanding from a low-profile configuration to an expanded configuration, each strut may have any suitable pattern.

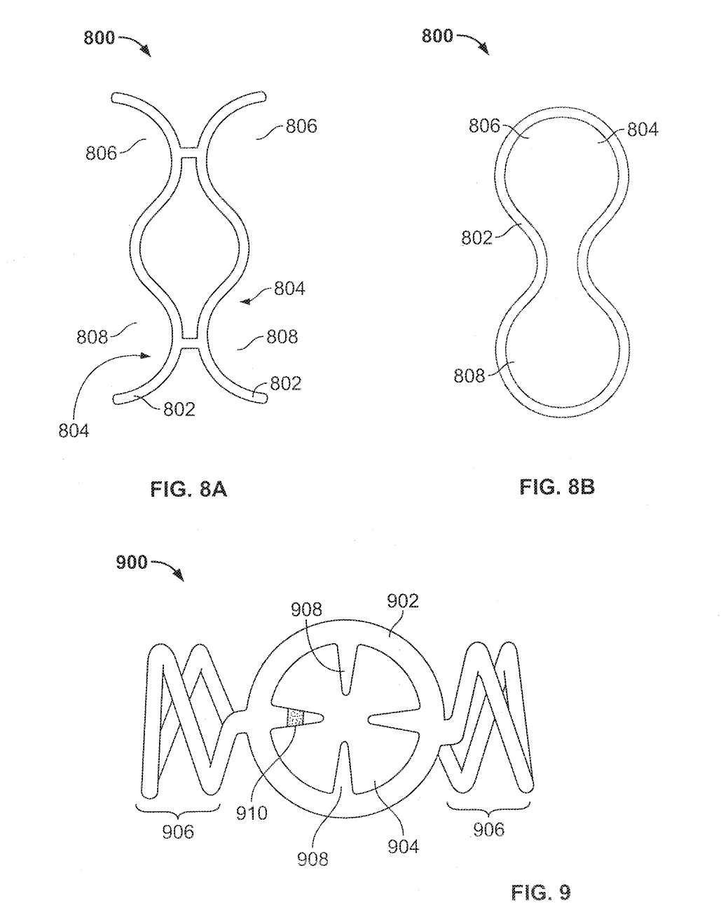

FIGS. 8A and 8B show a side view and a front view, respectively, of another variation of axial segment (800). As shown there, axial segment (800) may comprise two ring members (802), each ring member (802) defining an access port (804). When viewed from the front, as shown in FIG. 8B, access port (804) may be substantially peanut-shaped, and may comprise a first lobe (806) and a second lobe (808). When a treatment device is used to access port (804), the treatment device may enter the stent graft via any suitable portion of the access port (804) (e.g., first lobe (806), second lobe (808), combinations thereof, etc.). Additionally, while shown in FIGS. 8A and 8B as being the same size, first (806) and second (808) lobes may have different sizes.

In some variations, an access port may comprise one or more deflectable members. For example, FIG. 9 shows a front view of one such variation of axial segment (900). As shown there, axial segment (900) may comprise two saddle-shaped rings (902), defining access ports (904) and connected by two expandable portions (906). Additionally, saddle-shaped rings (902) may comprise one or more prongs (908) projecting into access ports (904). These prongs (908) may be flexible such that when a treatment device (not shown) or the like is advanced through one of the access ports (904), the treatment device may temporarily deflect one or more of the prongs (908) without moving or otherwise dislodging the stent graft. Additionally, the prongs (908) may be configured to return to their original positions once the treatment device is removed. Additionally, one or more of the prongs (908) may comprise one or more markers (910), but need not. It should be appreciated that any axial segment described here may comprise one or more flexible prongs, but need not.

In some variations, the stent framework may comprise one or more axial segments that do not comprise an access port. For example, in the stent framework (102) of stent graft (100) described in more detail above regarding FIGS. 1A-1D, second (110) and third (112) axial segments do not comprise an access port. These axial segments may still help to support the blood vessel and/or may help to anchor one or more portions of the stent graft relative to the vessel. Additionally while shown in FIGS. 1A-1D as comprising stent members (117) comprising a plurality of cells (119), it should be appreciated that the second (110) and third (112) axial segments may comprise any suitable stent members. In some variations, the stent members may comprise one or more patterned struts (e.g., zigzag or other meandering patterns).

In variations where the stent framework comprises two or more axial segments, each segment may or may not be connected to one or more additional segments. For example, in some variations, such as stent framework (102) described above in relation to FIGS. 1A-1D, the entire stent framework is formed as a monolithic structure from a single piece of material. In some of these variations, the stent framework may be cut (e.g., laser cut) from a cylindrical piece of material. In other variations, some or all of the axial segments may be formed as individual components and may subsequently joined (e.g., via chemical bonding, adhesive bonding, welding, or the like). In still other variations, individual components of the stent framework may not be directly connected, and instead may be held in place by the graft material, as will be described in more detail below.

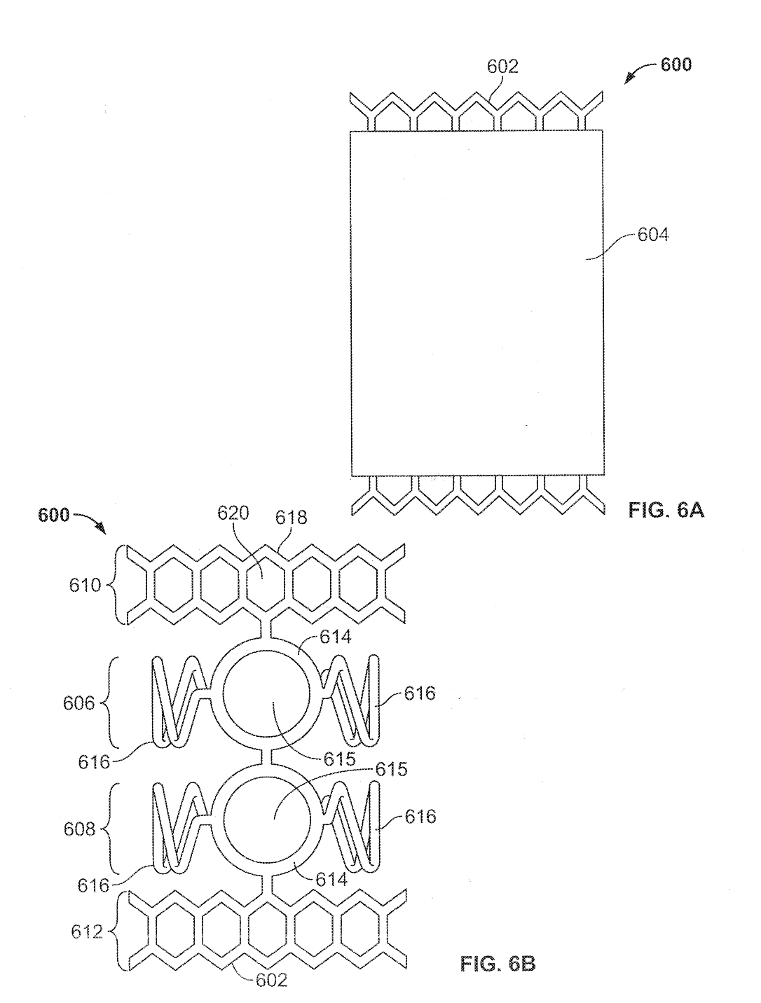

FIGS. 6A and 6B show one variation of stent graft (600). As shown in a front view in FIG. 6A, stent graft (600) may comprise stent framework (602) and graft material (604). FIG. 6B shows a front view of stent graft (600) without graft material (604). As shown there, stent framework (602) may comprise four axial segments (first (606), second (608), third (610), and fourth (612) axial segments). First (606) and second (608) axial segments each may comprise two saddle-shaped rings (614) connected via two expandable portions (616), as described in more detail above with respect to axial segment (200) shown in FIG. 2. Each saddle-shaped ring (614) may define an access port (615), as described in more detail above. Additionally, third (610) and fourth (612) axial segments each may comprise a stent member (618) comprising a plurality of expandable cells (620). Third (610) and fourth (612) axial segments may be configured to anchor stent graft (600) on either side of an opening (not shown) in a blood vessel. Additionally, it should be appreciated the four axial segments may comprise any suitable axial segment, such as those described above. While shown in FIG. 6A as entirely covering first (606) and second (608) axial segments and partially covering third (610) and fourth (612) axial segments, graft material (604) may cover any suitable portion or portions of the stent framework (602) as described in more detail above.

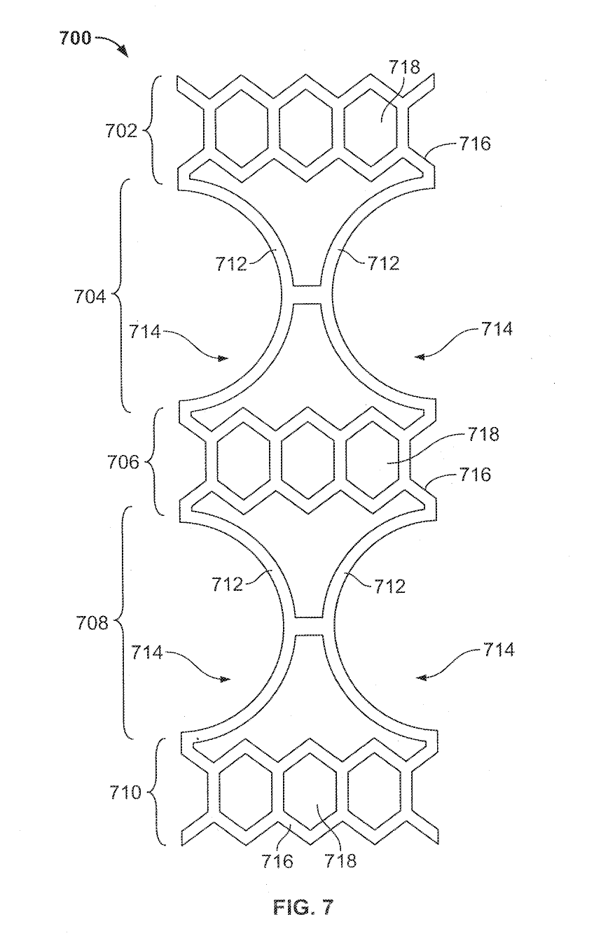

FIG. 7 shows,a side view of another variation of a stent framework (700) suitable for use with the stent grafts described here. As shown there, stent framework (700) may comprise, first (702), second (704), third (706), fourth (708), and fifth (710) axial segments. Specifically, second (704) and fourth (708) each may comprise two saddle-shaped rings (712), and each saddle-shaped ring (712) may define an access port (714), as described in more detail above. First (702), third (706) and fifth (710) axial segments each may comprise a stent member (716) having a plurality of expandable cells (718). First (702) and fifth (710) axial segments may be configured to anchor the stent graft proximally and distally of an opening in a vessel (not shown) as described in more detail above. Each axial segment of stent framework (700) may comprise any suitable combination of axial segments, such as those described above.

The stent framework and components thereof may be made from any suitable material or combinations of materials. In some variations, the entire stent framework may be made from the same material. In other variations, different portions of the stent framework may be made from different materials. The stent framework (or one or more portions thereof) may be biodegradable, bioabsorbable, or otherwise erodible, but need not be.

In some variations, one or more portions of the stent framework may comprise a shape-memory material. In some of these variations, one or more portions of the stent framework may comprise a nickel-titanium alloy (nitinol). Additionally or alternatively, one or more portions of the stent framework may comprise a copper-aluminum-nickel alloy, a copper-zinc-aluminum-nickel alloy, a shape memory alloys comprising zing, copper, gold, and/or iron, and combinations thereof. In some variations, one or more portions of the stent framework may comprise one or more polymers. Examples of suitable polymers include, but are not limited to, aliginate, cellulose, dextran, elastin, fibrin, hyaluronic acid, polyacetals, polyarylates (L-tyrosine-derived or free acid), poly(.alpha.-hydroxy-esters), poly(.beta.-hydroxy-esters), polyamides, poly(amino acid), polyalkanotes, polyalkylene alkylates, polyalkylene oxylates, polyalkylene succinates, polyanhydrides, polyanhydride esters, polyaspartimic acid, polybutylene diglycolate, poly(caprolactone), poly(caprolactone)/poly(ethylene glycol) copolymers, poly(carbonate), L-tyrosine-derived polycarbonates, polycyanoacrylates, polydihidropyrans, poly(dioxanone), poly-p-dioxanone, poly(epsilon-caprolactone), poly(epsilon-caprolactone-dimethyltrimethylene carbonate), poly(esteramide), poly(esters), aliphatic polyesters, poly(etherester), poly(ethylene glycol)/poly(orthoester) copolymers, poly(glutarunic acid), poly(glycolic acid), poly(glycolide), poly(glycolide)/poly(ethylene glycol) copolymers, poly(glycolide-trimethylene carbonate), poly(hydroxyalkanoates), poly(hydroxybutyrate), poly(hydroxybutyrate-co-valerate), poly(imino carbonates), polyketals, poly(lactic acid), poly(lactic acid-co-glycolic acid), poly(lactic acid-co-glycolic acid)/poly(ethylene glycol) copolymers, poly(lactide), poly(lactide-co-caprolactone), poly(DL-lactide-co-glycolide), poly(lactide-co-glycolide)/poly(ethylene glycol) copolymers, poly(lactide)/poly(ethylene glycol) copolymers, poly(lactide)/poly(glycolide) copolymers, polyorthoesters, poly(oxyethylene)/poly(oxypropylene) copolymers, polypeptides, polyphosphazenes, polyphosphoesters, polyphosphoester urethanes, poly(propylene fumarate-co-ethylene glycol), poly(trimethylene carbonate), polytyrosine carbonate, polyurethane, PorLastin or silk-ealastin polymers, spider silk, tephaflex, terpolymer(copolymers of glycolide,lactide or dimethyltrimethylene carbonate), and combinations, mixtures or copolymers thereof. Other suitable materials suitable for use in the stent framework include, but are not limited to, stainless steel, gold, tantalum, platinum, tungsten, niobium, ceramic, cobalt-chromium alloys, magnesium, aluminum, carbon fiber, combinations thereof and the like.

As mentioned above, the stent grafts described here generally comprise a graft material at least partially covering the stent framework. In some variations, the graft material covers the entire stent framework (e.g., both an interior and exterior surfaces of the stent framework). In some variations, such as stent graft (100) described in more detail above with respect to FIGS. 1A-1D, the graft material may cover only an outside surface of the stent framework. In other variations, the graft material may only partially cover the stent framework (102). For example, in variations where the stent framework of a stent graft comprises two or more axial segments, the graft material may fully cover some axial segments, but not cover (or partially cover) other axial segments. For example, in some variations where the stent framework comprises three axial segments (an intermediate segment and two end segments), the graft material may entirely cover the intermediate segment, but may only partially cover or not cover the two end segments. In others of these variations, the graft material may entirely cover the intermediate portion and one end segment, but may only partially cover or not cover the other end segment. Additionally, while graft material (104) shown in FIGS. 1A-1D above as being made from a single piece of material, it should be appreciated that in some variations the graft material (104) may be made from a plurality of pieces of material. In these variations, different pieces of graft material may be made from the same material, or may be made from different materials.

In variations where an axial segment comprises an access port, the graft material may fully or partially cover access port. In variations where the graft material covers an access port, entry into the stent graft through the access port (e.g., with a needle, catheter, or treatment device) may comprise puncturing, piercing, or otherwise penetrating the graft material. In some variations, the graft material may comprise one or more apertures or openings which may allow access through access port.

The graft material may be attached to the stent framework in any suitable manner. In some variations, the graft material may be bonded to, laminated on, or otherwise attached to a portion of the stent framework via one or more adhesives or chemicals. In other variations, the graft material may be attached to one or more portions of the stent framework via one or more mechanical attachment mechanisms such as a clip. In still other variations, the graft material may be sutured to one or more portions of the stent framework. In other variations, one or more portions of the stent framework may be sewn or otherwise contained in one or more pockets defined between two sections of graft material. In yet other variations, one or more stent members may be positioned to at least partially circumscribe the stent graft, and may act to hold the graft material in contact with the stent framework.

The graft material may or may not be biodegradable, bioabsorbable or otherwise erodible, and may be made from any suitable material or combination of materials. In some variations, at least a portion of the graft may be woven or braided. In these variations, the graft may be woven from any suitable fiber, strand, yarn, filament, or combinations thereof. In other variations, at least a portion of the graft may be non-woven, such as, for example, a solid film, sheet, or tube. The graft material may comprise a single layer, or may comprise a plurality of layers. In variations where the graft material comprises multiple layers, the layers may be made from the same material or materials, or may be made from different materials. Additionally, multiple layers may be connected in any suitable manner (e.g., via suturing, clamping, laminating, adhesive bonding, chemical bonding, or the like).

Examples of suitable graft materials include, but are not limited to collagen, polyethylene, polypropylene, polyacrylonitrile, cellulose, nylon, Dacron, polytetrafluoroethylene (PTFE), expanded polytetrafluoroethylene (ePTFE), polyurethane, polycarbonate-urethane, and polyethylene terepthalate. In some variations, the graft material may comprise one or more tissues or other extracellular matrixes. In some of these variations, the tissue may be derived from an autologous source. In other variations, the tissue may be derived from a xenologous source.

In some variations, one or more of portions of the graft material may comprise a coating. The graft may be coated using any suitable material, or materials, such as, for example, polyurethanes, silicones, one or more polymers (e.g., poly(ethylene glycol), poly(lactic acid), polyamides, PTFE, copolymers thereof), combinations thereof or the like. In other variations, one or more portions of the graft material may be seeded with one or more cells (e.g., one or more stem cells, one or more endothelial progenitor cells and the like.

In some variations of the devices described here, one or more portions of the stent graft (e.g., the graft material or the stent framework) may comprise one or more drugs or other bioactive agents. In some instances, one or more drugs or bioactive agents may be applied to one or more portions of the stent graft as a coating (e.g., by spray coating, dip-coating, brushing, or the like). In other variations, one or more drugs or bioactive agents may be directly incorporated into one or more portions of the stent graft, and may either diffuse therefrom or, in instances where one or more portions of the stent graft is biodegradable, may be released as those portions of stent graft biodegrade. In some variations, the graft may comprise one or more growth factors or other agents to help promote tissue ingrowth of tissue from the blood vessel. In other variations, the drug or bioactive agents may comprise one or more antiproliferative agents, one or more immunomodulating drugs, one or more antisclerosing agents, one or more anti-angiogenic agents, one or more thromboresistant agents, one or more anti-inflammatory agents, one or more genetic agents, one or more cell-regulating agents, derivatives, homologs, pharmaceutical salts, and combinations thereof.

As mentioned above, the stent graft may comprise one or more markers. These markers may be any suitable material capable of being viewed indirectly (e.g., via fluoroscopy, ultrasound, or the like). In some instances, one or more portions of the stent framework may be coated with or may otherwise comprises a radiopaque or echogenic material. Examples of suitable echogenic materials include, but are not limited to, barium sulfate, zirconium dioxide, cadmium, tungsten, gold, tantalum, bismuth, platinum, iridium, rhodium, or the like. In other variations, one or more radiopaque markers may be attached to a portion of the stent graft.

In some variations, one or more markers may help the user determine the rotational orientation of the stent graft within the vessel. This may help a user position the stent graft within a vessel, and may also help guide a user in inserting one or more needles, catheters, or treatment devices through an access port of the stent graft, as will be described in more detail below. In the variation of stent graft (100) shown in FIGS. 1A-1D above, the stent graft (100) comprises a plurality of markers (106) coating portions of stent framework (102). Specifically, two top markers (126) may be placed at the junctions between first (108) and second (110) axial segments, two bottom markers (128) may be placed at the junctions between the first (108) and third (112) axial segments, and two middle markers (130) may be placed at the junctions between the two saddle-shaped rings (114) of the first axial segment (110). When visualized, these markers (106) may present different patterns depending on the angle at which the stent graft is viewed. For example, when stent graft (100) is viewed from the front, as illustrated in FIG. 1B, the two top markers (126) may be substantially aligned, the two bottom markers (128) may be substantially aligned, and the two middle markers (130) may be out of alignment, such that the visible markers (106) are positioned in a diamond pattern. Conversely, when the stent graft (100) is viewed from the side, as illustrate in FIG. 1C, the two middle markers (130) may be substantially in alignment, while the top (126) and bottom (128) markers may be out of alignment. As a result, the visible markers (106) may present an x-shaped pattern. The visible markers (106) may change between these patterns as the stent graft (100) is rotated (or the point of view is rotated). Accordingly, during delivery, a user may view the relative positioning of the markers (106) to ensure that the stent graft (100) is placed in a particular rotational orientation within a blood vessel (e.g., to position one or more access ports (116) adjacent an existing vascular opening). Additionally, if a user needs to obtain subsequent access to the blood vessel, the markers (106) may indicate the position of the access ports (116).

In variations where an axial segment comprises a single access port, it may be difficult to determine during visualization whether the access port is pointing toward or away from the visualization device. To help alleviate this difficulty, the stent graft may comprise one or more markers that may indicate the directional orientation of the access port. For example, in the variation of axial segment (300) described above in relation to FIGS. 3A and 3B, the axial segment (300) may comprise three markers (308) (top marker (310), bottom marker (312), and side marker (314)). Visualization of the three markers (308) may allow a user to determine the rotational orientation of the stent graft, as immediately described above. Additionally, because the markers are asymmetric, a user may further be able to tell the direction in which the access port (302) is pointing. As shown in FIG. 3B, side marker (314) may be located on the right side of the access port (302) when the access port (302) is directed toward the visualization device. Conversely, the side marker (314) may be located on the left side of the access port (302) when the access port (302) is directed away from the visualization device.

In some variations one or more markers may comprise an asymmetric shape or pattern. These asymmetric markers may comprise any suitable shape or combinations of shapes, such as, for example, one or more arrows, letters, irregular shapes or the like. In these variations, the asymmetric marker may help a user to determine the rotational alignment of the stent graft. For example, in variations where a marker comprises an arrow shape, the arrow may point in one direction (e.g., to the right or left) when an access port is directed toward a visualization device, and may point in the opposite direction when the access port is directed away from a visualization device. In other instances, one or more markers may be formed into one or more letters, which may in turn spell a word. In these variations, the word may be readable when an access port is directed toward a visualization device, and may be unreadable/mirror-flipped when the access port is directed away from a visualization device, or vice versa. It should be appreciated that the stent graft may comprise any suitable number of markers, and these markers may have any suitable positioning in or on the stent graft.

The stent graft may have any suitable dimensions. Because the stent grafts may be expandable (e.g., self-expanding, balloon-expandable, or the like), the dimensions of the stent graft may change, depending whether the device is placed in a low-profile or an expanded configuration. In some variations, the stent graft may be at least about 6 mm in diameter when in an expanded configuration. In other variations, the stent graft may be at least about 7 mm in diameter when in an expanded configuration. In other variations, the stent graft may be at least about 8 mm in diameter when in an expanded configuration. In other variations, the stent graft may be at least about 9 mm in diameter when in an expanded configuration. In other variations, the stent graft may be at least about 10 mm in diameter when in an expanded configuration. Similarly, the stent graft may have any suitable length. In some variations, the stent graft may be between about 20 mm and about 40 mm. In other variations the stent graft may be between about 20 mm and about 30 mm, between about 30 mm and about 40 mm, between about 25 mm and about 35 mm, or the like. It should be appreciated that the dimensions of the stent graft may be chosen based upon the anatomy in which the stent graft will be delivered. For example, in some variations, the expanded diameter of the stent graft may be greater than the diameter of the vessel in which it will be placed, such that expansion of the stent graft within the vessel may press or hold the stent graft in place within the blood vessel.

In some variations, the stent grafts described here may comprise one or more sensors. For example, the stent grafts may comprise one or more flow or pressure sensors, such that a user may measure or otherwise determine the blood flow through the stent graft when placed in a vessel. Additionally, in some variations the stent graft may be configured to be retrievable, repositionable and/or removable after delivery to a vessel. In some variations, one or more grasping mechanisms may be used to move, remove, or otherwise reposition the stent graft. In other variations, the stent graft may comprise one or more tethers, sutures, wires or other similar structures for helping to move or reposition the stent. The tether may be pulled or otherwise manipulated (e.g., via one or more grasping mechanisms). In some variations, the tether may be at least temporarily attached to the stent framework. For example, in variations where a portion of the stent framework comprises a plurality of expandable cells, such as expandable cells (119) of stent member (117) as described above in relation to FIGS. 1A-1D above, a tether (not shown) or other suitable structure may be threaded through one or more cells of the stent framework. In other instances, the tether may be sewn into, tied to, or otherwise attached to the graft material.

### Methods

Also described here are methods for closing one or more openings in a vessel wall. In some variations, the methods described here are used to close one or more arteriotomies or other vascular openings formed prior to or during an endovascular procedure (e.g., EVAR or endovascular aortic valve replacement). In other variations, the methods described here may be used to close or seal one or more pseudoaneurysms or other iatrogenic holes (e.g., a retroperitoneal bleed) in a blood vessel. In some variations, the methods and devices described here may be used to seal an arteriovenous fistula. Generally, the methods described here may be used to seal one or more openings in an iliac artery (the common iliac artery, the internal iliac artery, or the external iliac artery) or the common femoral artery. The methods described here may be used to close openings created by large-French catheters and treatment devices, and in some instances may be used to close vascular openings greater than about 12 Fr (about 4 mm). In other instances, the methods may be used to close a vascular opening greater than about 15 Fr (about 5 mm). In other instances, the methods may be used to close a vascular opening greater than about 20 French (about 6.67 mm). In yet other instances, the methods may be used to close a vascular opening greater than about 27 French (about 9 mm).

Generally, the methods described here comprise advancing an introducer sheath comprising a balloon or other expandable member to a position upstream of the vascular opening. Once in place, the balloon may be expanded to occlude flow through the blood vessel. In variations where a catheter or treatment device is positioned through the vascular opening, the catheter or treatment device may be removed from the vascular opening. A delivery catheter may then be advanced through the sheath to a position near the vascular opening, and one or more closure devices may be delivered to the blood vessel to seal or otherwise close the opening. The closure device may be any suitable closure device, such as one or more of the devices described above. Once the opening has been closed, this closure may then be confirmed via angiography.

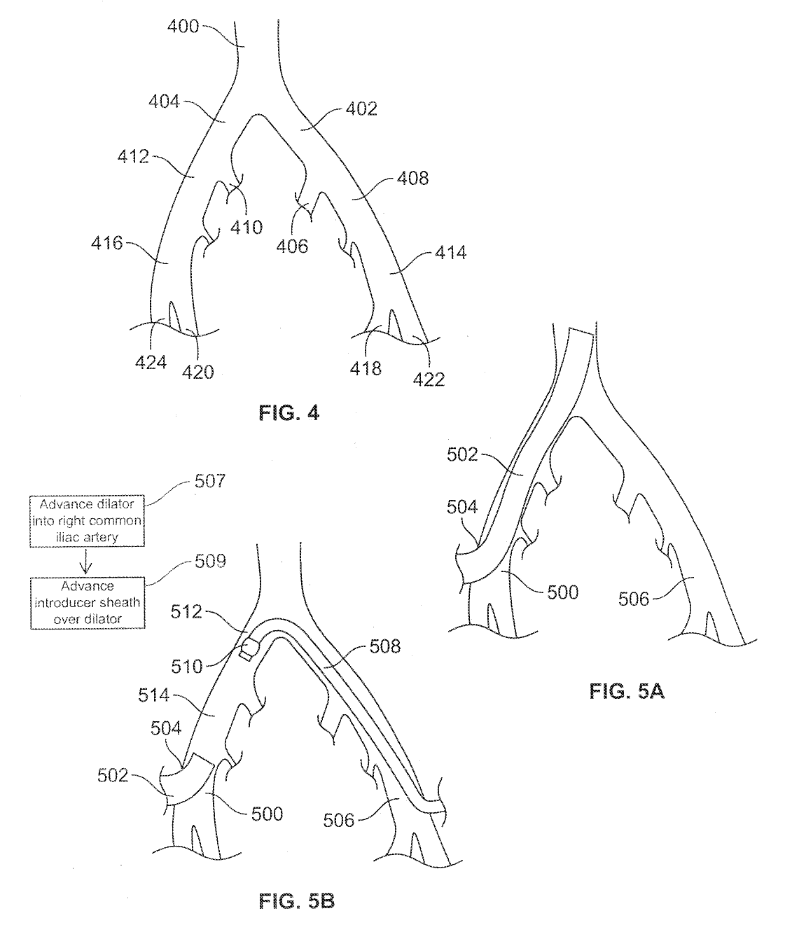

To aid in understanding of some the methods described here, FIG. 4 shows an illustrative depiction of some of the major arteries of the abdomen and legs. As shown there, the abdominal aorta (400) bifurcates around the level of the fourth lumbar vertebrae (not shown) into the left (402) and right (404) common iliac arteries. The left common iliac artery (402) later bifurcates into the left internal iliac artery (406) and the left external iliac artery (408). Similarly the right common iliac artery bifurcates into the right internal iliac artery (410) and the right external iliac artery (412). At or near the right and left inguinal ligaments (not shown) in the pelvis, the left (408) and right (412) external iliac arteries continues into the left (414) and right (416) common femoral arteries, respectively. Each of the common femoral arteries bifurcates into the deep femoral artery (labeled as (418) for the left and (420) for the right) and the superficial femoral artery (labeled as (422) for the left and (424) for the right).

Before initiating one of the closure procedures described here, it may be useful to determine and assess one or more relevant dimensions of the patient's anatomy. For example, in instances where a closure device will be placed inside of the common femoral artery, it may be desirable to measure the dimensions of the common femoral artery (e.g., the diameter of the artery and/or the length between the beginning of the common femoral artery and the bifurcation into the deep and superficial femoral arteries). Once the dimension of the common femoral artery has been determined (e.g., via angiography or the like), a user may pick a closure device that is properly sized to fit within common femoral artery.

As mentioned above, some of the methods described here may comprise sealing one or more vascular openings formed prior to or during an endovascular procedure. During these endovascular procedures, access to the vasculature is generally obtained via an opening formed in one of the common femoral arteries or one of the brachial arteries. A catheter or treatment device may be advanced through the opening, and may be further advanced to a target location to complete the endovascular procedure. Once the endovascular procedure has been completed, the opening may then be closed by one of the methods described here.

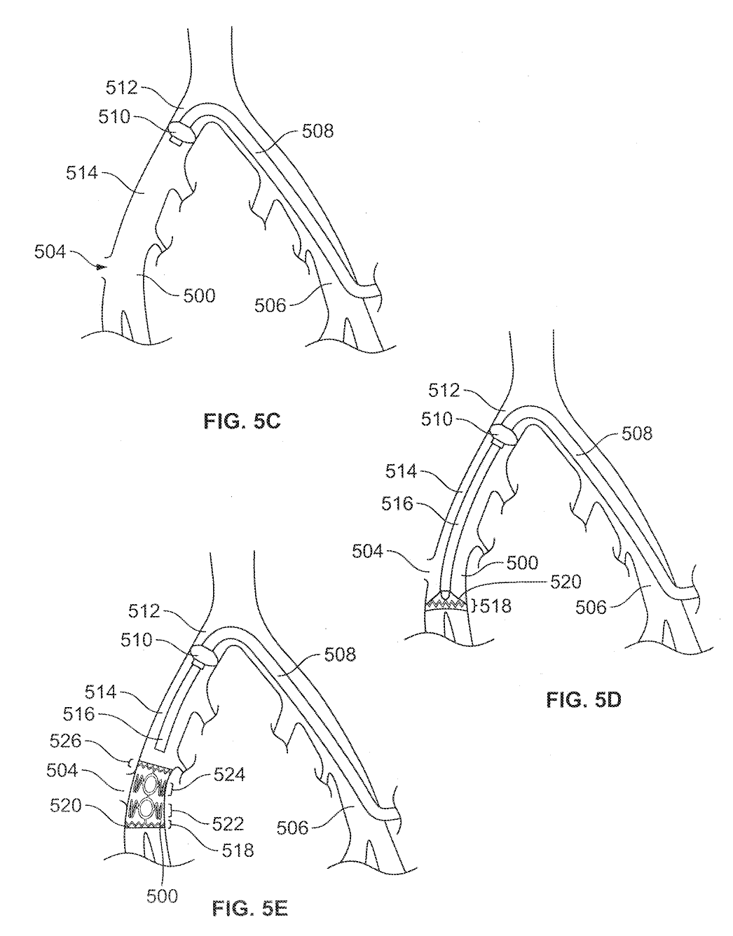

For example, FIGS. 5A-5E illustrate one method for closing a vascular opening formed in the right common femoral artery (500) during an endovascular procedure. As shown in FIG. 5A, treatment device (502) has been placed through an opening (504) in the right common femoral artery (500). It should be appreciated that although shown in FIGS. 5A-5E as accessing the vasculature via the right common femoral artery (504), the treatment device (502) may achieve vascular access through the left common femoral artery (506) as well. Following the completion of the endovascular procedure (e.g., endovascular aortic valve repair, endovascular aneurysm repair), the treatment device (500) may be partially withdrawn through the opening (504) and an introducer sheath (508) may be introduced into the vasculature via the contralateral common femoral artery (which in this variation is the left common femoral artery (506)) and advanced to position a balloon (510) or other expandable member upstream of opening (504). Balloon (510) may be compliant or non-compliant. While shown in FIG. 5A as being positioned in the right common iliac artery (512), it should be appreciated that the balloon (510) may be placed in any suitable position upstream of the opening (504) and the partially-withdrawn treatment device (502). For example, in some variations, the introducer sheath (508) may be advanced to position the balloon in the right external iliac artery (514). In other variations, the introducer sheath (508) may be advanced to position the balloon in the right common femoral artery (500).

It should be appreciated that the introducer sheath (508) may be advanced in any suitable manner. In some variations, the introducer sheath (508) may be at least partially advanced over a guidewire. In other variations, one or more curved dilators may be advanced into the right common iliac artery (507), and the introducer sheath (508) may be advanced over the curved dilator (509). It should also be appreciated that some or all of the method may be performed under fluoroscopic, ultrasound, or x-ray guidance. The introducer sheath may have any suitable diameter (e.g., about 6 French, about 7 French, about 8 French, or the like). Similarly, the introducer sheath may have any suitable length. For example, in some instances where the introducer sheath is advanced from a contralateral femoral artery, the introducer sheath may be at least about 40 cm, at least about 45 cm, at least about 55 cm, at least about 65 cm, or the like. In some instances where the introducer sheath is advanced from a brachial artery, the introducer sheath may be at least about 80 cm, at least about 90 cm, at least about 100 cm, at least about 110 cm, or the like.

Once in place, balloon (512) may be expanded to occlude blood flow past the balloon (512), and treatment device (502) may then be removed from the opening (504), as shown in FIG. 5C. Since the opening (504) is located downstream of balloon (512), occlusion of blood flow may allow the treatment device (502) to be removed from the opening (504) without substantial blood loss through the opening (504). Once the treatment device (502) has been removed, a delivery catheter (516) may then be advanced through a Lumen (not shown) of the introducer sheath (514), and may be used to deliver a closure device to close or seal the opening (504).

In some variations the delivery catheter (516) may deliver or otherwise deploy a stent graft, such as one or more of the stent grafts described above. In some of these variations, the delivery catheter (516) may be advanced downstream of opening (504), at which point a first axial segment (518) of stent graft (520) may be deployed downstream of the opening (504), as shown in FIG. 5D. In some variations, the delivery catheter (516) may be rotated to align the stent graft (520) relative to the vessel prior to deployment (e.g., to place one or more access ports in alignment with an anterior surface of the blood vessel as shown in FIG. 5E, to place one or more access ports in alignment with the opening (504), or the like). In variations where the stent graft (520) comprises one or more markers (not shown), such as those described in more detail above, these markers may be visualized to help deliver the stent graft (520) in a particular rotational and/or axial orientation. Similarly, one or more portions of the delivery catheter (516) may comprise one or more markers (not shown) that may be utilized to help position a stent graft with a certain rotational orientation. For example, the stent graft (520) may be positioned within the delivery catheter (516) such that a marker (not shown) of the delivery catheter (516) indicates the rotational position of one or more access ports. A user may then align the marker of the delivery catheter (516) with a surface (e.g., the anterior surface) of a blood vessel or one or more openings in a blood vessel, and the stent graft (520) may be delivered such that one or more access ports are aligned with that surface or opening. Any suitable portion or portions of the delivery catheter (516) may comprise one or more markers (e.g., the catheter body, a nosecone (not shown), combinations thereof, and the like), and the one or more marker may comprise any suitable marker or markers, such as those described above. The delivery catheter (516) may then be withdrawn to deliver a second (522) and third (524) axial segment adjacent the opening (504), and a fourth axial segment (526) upstream of the opening (504), such that stent graft (520) covers and/or seals opening (504), as shown in FIG. 5E. Although stent graft (520) shown in FIGS. 5D and 5E comprises first (518), second (522), third (524), and fourth (526) axial segments, the stent graft may comprise any suitable number and configuration of axial segments, as described in more detail above.

Once the stent graft (520) has been deployed to close opening (504), closure may be confirmed via angiography or another suitable technique. In some variations, confirming closure of the opening (504) may comprise deflating the balloon (510). Additionally, one or more radiopaque dyes (not shown) may be introduced into the vasculature (e.g., by delivery catheter (516), introducer sheath (508), or another suitable device), and fluoroscopy may be used to look for dye leaking or otherwise passing through opening (504). Additionally, one or more treatment devices may have inadvertently formed an additional opening, cut or hole in one or more of the blood vessels, and the radiopaque dye may be used to detect these additional openings. If necessary, balloon (510) may be re-inflated, and one or more additional closure devices may be delivered through the introducer sheath (508) and/or the delivery catheter (516), to ensure closure of any of these openings. In some of these variations, delivery catheter (516) may be withdrawn through introducer sheath (508), and a second delivery catheter (not shown) may be advanced through introducer sheath (508) to deliver a second closure device (not shown). Once the opening (504) (and any other openings) have been properly closed, the introducer sheath (508) and delivery sheath may be removed from the body.

While the methods described above in relation to FIGS. 5A-5E are utilized to close an opening in the common femoral artery, it should be appreciated that similar approaches may be used to close an opening in one of the iliac arteries (the common iliac artery, the internal iliac artery, or the external iliac artery). Additionally, similar approaches may be used to close one or more pseudoaneurysms or other iatrogenic holes. The devices and methods described here may also be utilized to close one or more openings in one or more veins. It should also be appreciated that any suitable closure device may be delivered by the devices described here, and that the devices described here may be deployed by any suitable method.

What is claimed is:

1. A method of closing an opening in the common femoral artery, external iliac artery, internal iliac artery, or common iliac artery, comprising: advancing a introducer sheath to a position upstream of the opening, wherein the introducer sheath comprises an expandable member; expanding the expandable member to occlude blood flow past the expandable member; advancing a delivery catheter through the introducer sheath to a position near the opening; and delivering a closure device to close the opening.

2. The method of claim 1 further comprising introducing a dilator into a contralateral femoral artery and advancing the dilator into the common iliac artery, and wherein advancing the introducer sheath comprises advancing the introducer sheath over the dilator.

3. The method of claim 1 wherein the closure device comprises a stent graft, the stent graft comprising a stent framework having a first axial segment and a graft material at least partially covering the stent framework, and wherein the first axial segment comprises an access port in a side of the stent framework, the first access port is sized and configured to receive a treatment device therethrough.

4. The method of claim 1 wherein the expanding the expandable member comprises expanding the expandable member in the common iliac artery.

5. The method of claim 1 wherein expanding the expandable member comprises expanding the expandable member in the external iliac artery.

6. The method of claim 1 wherein expanding the expandable member comprises expanding the expandable member in the common femoral artery.

7. The method of claim 1 further comprising introducing the introducer sheath into a brachial artery.

8. The method of claim 1 further comprising contracting the expandable member and confirming closure of the opening.

9. The method of claim 8 further comprising re-expanding the expandable member and repositioning the closure device.

10. The method of claim 8 further comprising re-expanding the expandable member and delivering a second closure device to the opening.

11. A method for closing an opening in a blood vessel, the opening having a treatment device placed therethrough, the method comprising: partially withdrawing the treatment device from the blood vessel; advancing an introducer sheath to a position upstream of the opening, the introducer sheath comprising an expandable member; expanding the expandable member to occlude blood flow through the blood vessel; removing the treatment device from the blood vessel; and delivering a closure device to the blood vessel to close the opening.

12. The method of claim 11 wherein the blood vessel is a common femoral artery.

13. The method of claim 12 wherein advancing the introducer sheath comprises advancing the introducer sheath through a contralateral femoral artery.

14. The method of claim 11 wherein delivering the closure device comprises advancing a delivery catheter through the introducer sheath, and delivering the closure device from the delivery sheath.

15. The method of claim 11 wherein the closure device comprises a stent graft, the stent graft comprising a stent framework having a first axial segment and a graft material at least partially covering the stent framework, and wherein the first axial segment comprises an access port in a side of the stent framework, the first access port is sized and configured to receive a treatment device therethrough.

16. The method of claim 15 further comprising aligning the stent graft such that the first access port is placed adjacent to an anterior surface of the blood vessel.
